(19) United States
(12) Patent Application Publication
(10) Pub. No.: US 2022/0265707 A1
(43) Pub. Date: Aug. 25, 2022

(54) ANTIMICROBIAL NANO-SURFACTANT AND METHODS

(71) Applicant: Exothule Corporation, Irvine, CA (US)

(72) Inventor: Peter Butzloff, Walnut, CA (US)

(21) Appl. No.: 17/741,462

(22) Filed: May 11, 2022

Related U.S. Application Data
(63) Continuation of application No. PCT/US20/23024, filed on Mar. 16, 2020.
(60) Provisional application No. 62/966,010, filed on Jan. 26, 2020.

(51) Int. Cl. A61K 33/44; A01N 59/26; A23L 33/00; A23L 33/10

(52) U.S. Cl. CPC A61K 33/44; A01N 59/26; A23L 33/40; A23L 33/10; A01P 1/00

(57) ABSTRACT

An antimicrobial composition of buckminsterfullerene with saponified phosphorus acid functional groups is provided to disassemble or make virus particles inert, and to inhibit viral and fungal proteases using catalytic desulfurization. This composition is formulated to prevent or to treat novel corona viruses including emerging strains of SARS-Cov-2, as well as fungal pathologies such as valley fever and respiratory ailments such as chronic obstructive pulmonary disorder (COPD) and pneumonia. Virus particles are implicated in the development of cancers. The antiviral properties further enable the composition to prevent conditions leading to uncontrolled cellular proliferation, neoplasms, degenerative malignancy, and to help treat chronic inflammatory diseases associated with or leading to induce cancer in virus infected cells. The composition can be produced at low temperatures through reactive shear mixing. Delivery methods include ingestion, topical application, inhalation, or injection when used as a medicament or as a food supplement.

### CROSS-REFERENCE TO RELATED APPLICATION

This application is a continuation of International Application PCT/US20/23024 filed on Mar. 16, 2020 which claims the benefit of U.S. provisional patent application 62/966,010 filed on Jan. 26, 2020, both of which are incorporated herein by reference in their entireties. This application is also related to US application 17/xxx,xxx filed on even date herewith, (attorney docket number 10624.02US) and titled “ANTIMICROBIAL NANO-DELIVERANT AND METHODS” which is also a continuation of International Application PCT/US20/23024 and also incorporated herein by reference in its entirety.

### BACKGROUND

### 1. Field of Invention

The present invention is a cation hopping and size constrained composition of buckminsterfullerene bonded with sodium phosphonate pendant functional groups functioning as a nano-surfactant, with methods of use to convey or deliver therapeutic molecules to prevent or to treat chronic respiratory illnesses such as obstructive pulmonary disorder (COPD), and to help treat uncontrolled cytokine storm arising from inflammatory reaction to invasive microbes including viruses, fungi, and some types of infectious bacteria. These same properties promote usage for treating microbially induced cognitive decline and to promote preventative health to protect against microbial invasion of neural tissues. Delivery methods include ingestion, topical application, inhalation, or injection when used as a medicament or as a food supplement to maintain or re-establish benign healthy acquired immune homeostasis.

### 2. Background Art

All present state of the art antiviral compounds and compositions have been directed at the exterior proteins and surfaces of the protective protein shell of latent virus particles, or at the inhibition of the digestive proteases of the active molecular machinery of such viruses. For the most part, fungal pathogens often host viral particles, leading to synergy between these types of microbes. It becomes increasingly difficult to combat one of these organisms when one or more of them is pathogenic. It is also possible for various species of otherwise helpful and commensal bacteria to sometimes become infected with pathogenic viral strains that are almost impossible for either the innate or the acquired immune system to identify and destroy. One type of auto immune response is to create reactive oxygen and reactive nitrogen species, however when the pathogenic infection becomes chronic, these types of somatic responses to pathogenic microbial invasion serve to cause long term inflammation that degrades the overall health of the body, and may eventually lead to death if left untreated.

Anti-inflammatory compositions targeting the well-known cellular roles of NADPH and superoxide dismutase (SOD) at the mitochondria have been or are continuing to be developed to treat diverse pathological cell conditions leading to inflammation without significantly ridding the body of microbially induced disease. Such conditions include but are not limited to cancer, cognitive decline, arthritis, diabetes, vascular disease, neurological disease, and colitis. Nowadays, multiple functions are designed into such substances to allow anti-Tumor Necrosis Factor Alpha (anti-TNFα), and anti-inflammatory interleukins such as anti-IL-6, and anti-IL-1 therapies simultaneously. Such multifunctional compositions are being tested in clinical trials for efficacy with a spectrum of outcomes. These substances are also being considered as interventions in the aging process to evaluate if any of the treatments might improve the health span of aging individuals. Unfortunately, many of these treatments or compositions are poorly bioavailable, being soluble in water and being unable to pass cellular membranes or being oil soluble and poorly able to be carried by the blood in the circulatory system. Typical drug loading of about 10% is achieved for nanoparticles of a narrow size distribution around an average size of about 100 nanometers when encapsulated in water soluble polymer micelles, suggesting complete dispersibility of such substances, but when doing so, this results in the substantial or complete masking of the therapeutic agent, along with poor targeting to the microbes that are invasive to the cells of the body.

Several different types of genetic predispositions are known to induce traumatic muscle cell injury termed myopathy, where the lack of ability to produce a protein is implicated. In one such type of genetic deficit, the lack of an ability to produce dystrophin leads to the illness called Duchenne muscular dystrophy (DMD) and the related Becker muscular dystrophy (BMD), which can result in respiratory failure and pneumonia that results in the early death of the individual in childhood. One research effort recently released a state-of-the-art attempt to treat DMD with a potassium substituted fullerene phosphonate. Muscle cells require the controlled release of sodium ions and to a different extent, potassium ions. The lack of certain proteins causing myopathy is most certainly related to pathological ion channel control failure. However, while the present medical attention is directed to potassium ion channelopathies, there is no corresponding report to address the sodium ion channelopathies in muscular myopathies. This conceptual dissonance is not surprising, as the expertise in one type of channelopathy are often not correlated to treat disease pathologies in another type of gated ion channel, as these have very different medical functions and significantly different drug targets.

All microbes constantly evolve, leaving the medical field new challenges to maintain effective countermeasures against suddenly altered pathogens. The dual bioavailability and pathogen targeting problems are part of the significant obstacles to commercial and medical success of the latest multifunctional antibiotic and antiviral prophylactic and therapeutic compositions. This process has been increasingly apparent with the onset of ‘long-covid’ in which compromised immunity leads to colonization and inflammation of the brain by various strains of SARS-Cov-2. Also known as covid-19 for the year in which the pandemic started, the evolved strains of this virus in combination with less aggressive endemic viruses such as herpes simplex virus-1 (HSV-1) and commensal zoonotic fungal spores such as Candida albicans, are now increasingly causing what is commonly known as ‘brain fog’ and catastrophic breakdown in rationality exhibited as ‘road rage’ and other incomprehensible behavioral changes and symptoms throughout a portion of the global population. There are no proactive strategies to eliminate either the symptoms or the associated pathologies for these conditions in the present state of the art.

What is therefore needed is a novel therapeutic strategy or unique material used to confer microbial protection in advance to protect against evolving and future pathogens even before they have developed new infectivity or altered biochemistries. A noble medical objective has been to strive toward some generic method to prevent, mitigate, or reverse the onset of drug resistant pathogens and illness before irreversible or life-threatening damage progresses within infected individuals. Desirably, such an antimicrobial treatment should include a means to cross the blood-brain-barrier (BBB) to confer prophylactic maintenance or enhancement of cognitive function well into old age. It is believed the present invention provides such a composition, having a biological and electrochemical design to confer multiple therapeutic and prophylactic functions. The use of different carrier formulations described herein enables appropriate methods of administration for this nanoparticle composition.

### SUMMARY OF THE INVENTION

This invention is a cluster of nanoparticles comprising carbon fullerenes covalently derivatized with phosphonates having oxidation state of three, where this substance is saponified with a cationic sodium, or like alkali earth element, that is capable of reversibly shuttling between the oxygen groups of the phosphonate and the bare aromatic carbon face of the buckminsterfullerene by means of pi-cation bonding. The pendant acid phosphonates are saponified or neutralized with cations, preferably sodium, to form disodium phosphonate groups having a surfactant nature and also having a viral and fungal protease inhibiting function via the phosphonate sulfurization reaction. This composition also possesses properties which reflect the singular free radical scavenging chemical function of fullerenes, a viral capsid and spike protein disassembly function, and a biosurfactant function that couples with pulmonary surfactant to reduce its viscosity and enhance cytokine and chemokine solubilization for redirection away from airway passages and to remediate capillary blood vessel clotting reactions.

The result of these combined functions is to allow time for the proper management of the innate immune response so that the acquired immunity can be established as each new variant of rapidly evolving microbes appears in the body to re-establish functional immune homeostasis.

An aspect of the present composition is that the molecular structure of buckminsterfullerene sodium phosphonates is such that it produces reversible hopping pi-cation bonded adducts between the sodium cations of the hydrophilic phosphonate groups and the hydrophobic aromatic regions of the C60 carbon which functions as a hydrophobic penetrant to van-der-Waals charge stabilized viral proteins.

In another related aspect, the sodium cations on the phosphonate groups are immediately charge attracted to highly negatively charged exterior hydrophilic regions at the edges of viral capsid glycoprotein plates and at the outside of spike glycoproteins as a nano-surfactant material that is able to deconstruct the viral structure by means of the sodium that is pi-cation bonded to the hydrophobic carbon face of the C60 pendant group.

In a related aspect, sodium cations with pi-cation bonds to a buckminsterfullerene molecule have sufficiently small dimensions to displace the chloride ion charge pinning regions in viral spike glycoproteins to allow their immediate disassembly, thereby releasing sodium chloride.

In a related aspect, the fullerene sodium phosphonate can perform viral protein capsid van-der-Waals charge disruption, thereby functioning to explode and denature virus particles by taking apart their protective protein shells and exposing their viral RNA for removal by the immune system before these viral particles can infect a cell.

In another aspect, the fullerene sodium phosphonates provide nano-surfactant chaperoning to allow monoclonal antibodies and therapeutic drugs having marginal bioavailability or poor cell membrane penetration to become sandwiched and carried for optimal delivery to their appropriate sites of therapy.

In a related aspect, the reversible hopping of the pi-cation bonds between hydrogen and sodium ions and the core fullerene C60 provides a novel in-vivo stability for resistance to the digestive process.

In another aspect, the composition of fullerene sodium phosphonate is used to treat COPD by inhalant delivery to the lungs and airways of a patient (a human person or animal) in need of treatment.

In a related aspect, the fullerene sodium phosphonate composition is used to treat fungal lung infections such as black fungus or valley fever of the lungs and airways, again by inhalant delivery to a patient that is experiencing this type of chronic inflammatory bronchitis.

In another aspect, the sodium phosphonates pendant from the fullerene serve to inhibit viral proteases as well as to remove their catalytic protein digestion capability by irreversibly bonding to and extracting the catalytic sulfur atom from the protease. This immediately halts the pathological impact of malignant, cancer-causing viruses to deconstruct, infect, and usurp the function of otherwise healthy somatic cells. This can also immediately halt a pathological fungal invasion of sensitive organs and tissues.

In another aspect, the surfactant properties of the fullerene sodium phosphonate allow it to diffuse throughout the extremely small dimensions of the hydrophobic regions within viruses. Larger molecules such as organic substituted bisphosphonates are unsuitable to fit inside these viral structures. Cations with dimensions greater than that of sodium, such as potassium, are unsuitable because these ions are too large to perform the viral disassembly function for substantially most types of virus particles.

In a related aspect, the sodium cations that reversibly hop to and from the phosphonate functional groups are the only ions sufficiently small, and of high enough charge density, to enable the fullerene mediated pi-cation mechanism that is required to implement the viral disassembly process using van-der-Waals charge disruption.

In another aspect, the core fullerene molecule functions to disassemble detrimental van-der-Waals mediated hydrophobic charge zippers at usurped cell membranes while decorating and defusing them with nano-surfactant deposits that interfere with and halt the function of viral replication platforms. This action coats and destroys the zippering mechanism of viral protein glycoprotein filaments by masking their hydrophobic van-der-Waals mediated charge zippers. This action of de-zippering the viral glycoprotein filaments is achieved by coating their zippering regions with a multiplicity of fullerene sodium phosphonates.

In another aspect, the chelation ability of the fullerene sodium phosphonate allows the molecule to function as a free radical recombination and detoxification center, thereby reducing inflammation and serving to boost innate immunity while reducing the tendency for excessive cytokine storms and chemokines to inflict damage on tissues.

In another aspect, the chelation ability of fullerene sodium phosphonates allows one of more of these molecules to form a complex with a therapeutic drug molecule. The drug-FSP complex is coupled by hydrophobic van-der-Waals attractive forces of the carbon atoms of the C60, and by hydrophilic ionic charged groups of the therapeutic drug molecule that forms an attraction to opposing charged groups on the phosphonate groups, termed a ‘counter-ion’ or faradic charge coupling.

In a related aspect, the method of making chelated drug-FSP using both counter-ions and van-der-Waals bonding is substantially enhanced by reactive chemo-electric shear mixing, also known as chemical electric reaction shear mixing, wherein the use of electric forces expedites a low temperature reaction process and increases the rate of that reaction. This method is used to minimize damage to the therapeutic molecule by the shearing process performed on an expensive antibody or drug molecule to be delivered by means of the FSP-drug complex.

In another aspect, the fullerene sodium phosphonate composition is heated to form a nano aerosol for the purpose of immediate aspirated delivery to the lungs, thereby providing access to the blood system for rapid release of the administered inhalant composition.

In a related aspect, the sodium fullerene phosphonates serve as a viscosity reducing agent that serves to reduce the viscosity of pulmonary surfactant to enable the clearing of the lungs of patients with respiratory disease such as pneumonia of any type.

In another aspect, Duchenne muscular dystrophy (DMD) resulting in respiratory failure and death by pneumonia from sodium ion channelopathy is significantly remediated by the administration of fullerene sodium phosphonate, wherein the role of sodium ion hopping is to prosthetically replace the role of the dystrophin protein to regulate sodium ions. This therapy can stabilize the homeostasis of sodium regulation in heart, respiratory, and other muscles affected by myopathy without resort to what are presently considered dangerous and irreversible genetic alterations.

These and other advantages of the present invention will be further understood and appreciated by those skilled in the art by reference to the following written specifications, claims, and appended drawings.

Some embodiments are described in detail with reference to the related drawings. Additional embodiments, features, and/or advantages will become apparent from the ensuing description or may be learned by practicing the invention. In the drawings, which are not to scale, like numerals refer to like features throughout the description. The following description is not to be taken in a limiting sense but is made merely for describing the general principles of the invention.

### BRIEF DESCRIPTION OF THE DRAWINGS

Preferred embodiments of the invention will now be described, by way of example, with reference to the accompanying drawings, in which:

FIG. 1 illustrates the molecular structures of an exemplary chemical reaction with buckminsterfullerene to synthesize the antiviral antifungal phosphonate nanoparticle molecular structure.

FIG. 2 illustrates the molecular structures of an exemplary chemical reaction with fullerenol to synthesize the antiviral antifungal phosphonate nanoparticle molecular structure.

FIG. 3 illustrates an inset view of one portion of a molecular structure of buckminsterfullerene.

FIG. 4 illustrates one portion of a molecular structure of buckminsterfullerene disodium phosphonates to produce reversible hopping pi-cation bonded adducts.

FIG. 5 illustrates the nano-surfactant chaperoning of an antibody protein sandwiched between the molecular structures of FDSP.

FIG. 6 illustrates the viral protein capsid van-der-Waals charge disruption function.

FIG. 7 illustrates the shape of an influenza or a corona virus having spike glycoproteins suitable for van der Waals charge disruption using the sodium phosphonate nanoparticle molecular structure.

FIG. 8 illustrates a side view of the chloride ion stabilized viral spike glycoprotein prior to the designed nano-surfactant mediated van-der-Waals charge unzippering required to unravel the globular capsid protein shell.

FIG. 9 illustrates a section view of the chloride ion ejection from the hydrophobic region of a viral spike glycoprotein via van-der-Waals mediated charge unzippering and surfactant mediated properties.

FIG. 10 illustrates reconstruction and recovery of zippered cell membranes usurped and repurposed as viral replication platforms.

FIG. 11 illustrates the desulfurization reaction between sodium phosphonates to extract a protease catalytic sulfur from a cysteine or other sulfur containing amino acid.

FIG. 12 is a flowchart showing exemplary steps to prepare vapor inhalant formulations of fullerene sodium phosphonates.

FIG. 13 is a flowchart showing exemplary steps to prepare oral and topical formulations of fullerene sodium phosphonates.

FIG. 14 illustrates experimental FTIR test data of fullerene sodium phosphonate.

FIG. 15 illustrates experimental negative mode MALDI-TOF mass spectrograph data for five and ten sodium phosphonate derivatives of C60.

FIG. 16 illustrates the integrated peak area function for the data of FIG. 14.

FIG. 17 illustrates experimental negative mode MALDI-TOF mass spectrograph data for five, ten, and fifteen sodium phosphonate derivatives of C60.

FIG. 18 illustrates the integrated peak area function for the data of FIG. 16.

FIG. 19 exemplary methods to administer formulations to contain and deliver fullerene sodium phosphonate.

FIG. 20 illustrates inhalant fullerene sodium phosphonates entering the lungs

FIG. 21 illustrates an exemplary method of personal inhalant administration of a nano-aerosol formulation.

FIG. 22 is a flowchart showing exemplary steps to prepare fullerene sodium phosphonate in blood plasma for medical injection.

FIG. 23 illustrates alternative fullerene structures for pi-cation hopping enabled sodium phosphonates.

FIG. 24 is a flowchart showing exemplary steps to prepare drug complexed FSP.

FIG. 25 is a flowchart showing exemplary steps to prepare bis- and tris-FSP.

### DETAILED DESCRIPTION OF THE INVENTION

The following detailed description, taken in conjunction with the accompanying drawings, is merely exemplary in nature and is not intended to limit the described embodiments or the application and uses of the described embodiments. Any implementation described herein as “exemplary” or “illustrative” is not necessarily to be construed as preferred or advantageous over other implementations.

Furthermore, there is no intention to be bound by any expressed or implied theory presented in the preceding technical field, background, brief summary or the following detailed description. It is also understood that the specific devices, systems, methods, and processes illustrated in the attached drawings, and described in the following specification, are simply exemplary embodiments of the inventive concepts defined in the appended claims that there may be variations to the drawings, steps, methods, or processes, depicted therein without departing from the spirit of the invention. All these variations are within the scope of the present invention. Hence, specific structural and functional details disclosed in relation to the exemplary embodiments described herein are not to be interpreted as limiting, but merely as a representative basis for teaching one skilled in the art to variously employ the present embodiments in virtually any appropriate form, and it will be apparent to those skilled in the art that the present invention may be practiced without these specific details.

Various terms used in the following detailed description are provided and included for giving a perspective understanding of the function, operation, and use of the present invention, and such terms are not intended to limit the embodiments, scope, claims, or use of the present invention.

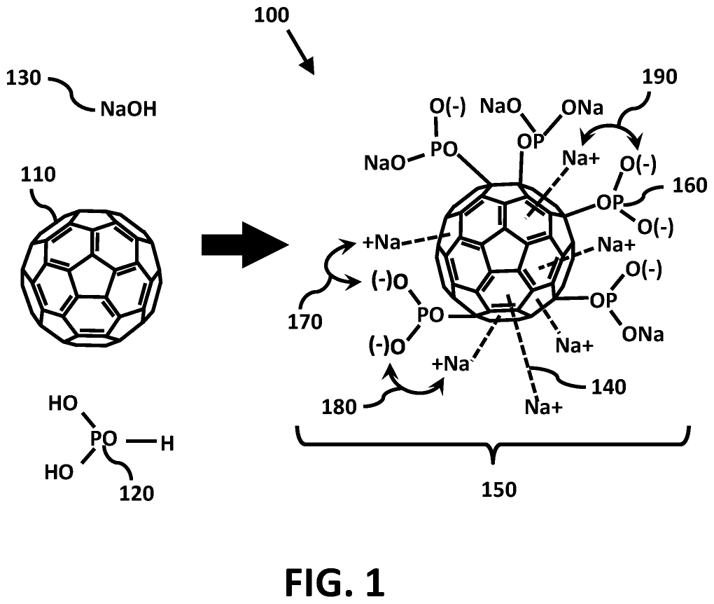

FIG. 1 is an illustration of an exemplary chemical reaction 100 to synthesize fullerene sodium phosphonates. Buckminsterfullerene is a molecule in the shape of a spherical chemical cage representing 60 aromatic carbon atoms with formula C60, also known herein as the fullerene molecule 110. The scientific literature reports a measured single pristine fullerene C60 molecule has a physical particle size about 0.7 nm and a van der Waals diameter of 1.1 nm. The molecular structure of phosphorous acid 120 contains a phosphorous atom with the oxidation state of 3. Fullerene and phosphorous acid are reacted in chemical reaction 100 to form fullerene sodium phosphonate 150 that can then be neutralized or saponified by further reaction with sodium hydroxide (NaOH) 130 to provide the reaction indicated by the direction of the large black arrow. The sodium ion (Na+) has a diameter of 116 picometers and can reversibly form cation-pi stacking bonds with the aromatic carbon face of the fullerene functional group as indicated by the dashed line 140 for a multiplicity of cation-pi bonded sodium ions. Cation hopping may leave some phosphonate functional groups temporarily bare of sodium cations to expose negative charges on the distal oxygen atoms 160. Examples of reversible sodium ion hopping are indicated by the curved double headed arrows 170, 180, 190. The distance of this hopping is less than 5 nanometers to allow the necessarily facile sodium charge hopping function enabled by sodium phosphonates in this molecular structure to enter virus particles or their proteases. Virus particle spike glycoproteins have a dimensionally confined gallery cavity of about 1.5 to 2 nanometers. To allow facile entry to the confined geometry of viral structures and enable fullerene phosphonate use as a viral penetrant, the hydrophobic fullerene carbon face enters the hydrophobic region of the virus structure with the pi-cation stabilized sodium ion to react with the viral chloride charge pinning ion and destabilize it to enable automatic disassembly of the virus. Proximity of the sodium phosphonate group to the fullerene allows the facile reversible sodium cation hopping to the fullerene by means of pi-cation bonding. Alternative fullerene phosphonate structures can become acceptable replacements for use in the present composition, provided that facile sodium charge hopping is provided from the sodium phosphonates to the hydrophobic fullerene group. The composition of fullerene sodium phosphonates provides hydrophobic viral protein penetration, facile faradic nano-surfactant function by means of sodium phosphonates proximal to the fullerene group and proximal van der Waals induced charge disruption ability of the sodium ion decorated fullerene face of the fullerene sodium phosphonate, according to these teachings.

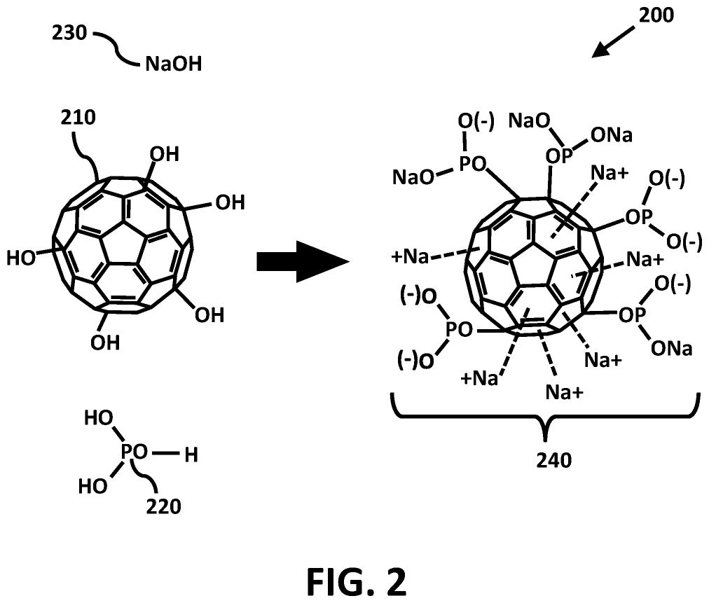

FIG. 2 is an illustration of an exemplary alternative chemical reaction 200 to synthesize antiviral antifungal fullerene sodium phosphonates. In this case, polyhydroxylated fullerenes termed fullerenols 210 may be used as the starting material. The number of hydroxyl groups (OH) on the fullerenol 210 can vary from at least one to as many as 26 without being considered a toxic material; five hydroxylations are shown for the purpose of this example. It is noted that the use of stoichiometry by counting the number of hydroxylations derivatized onto the fullerenol starting material is to match with the number of phosphonates for the purpose of the chemical reaction to make fullerene sodium phosphonate. In some embodiments, there is no residual, impurity, or excess polyhydroxylated fullerene remaining after the use of this starting material to compromise the purity of the resulting fullerene sodium phosphonate. The fullerenol 210 is reacted with phosphorous acid 220 using reactive shear mixing at 90° C. for 25 minutes to form the fullerene phosphonate intermediate (not shown). On allowing this mixture to cool, sodium hydroxide is added using reactive shear mixing at 90° C. for 25 minutes to form the fullerene sodium phosphonate 240. In various embodiments the stoichiometric ratio is two sodium atoms per phosphonate group where at least one sodium ion is able to achieve the requisite sodium hopping mechanism at a span of less than 5 nanometers required for the functional operation of this nanoparticle molecular structure according to these teachings.

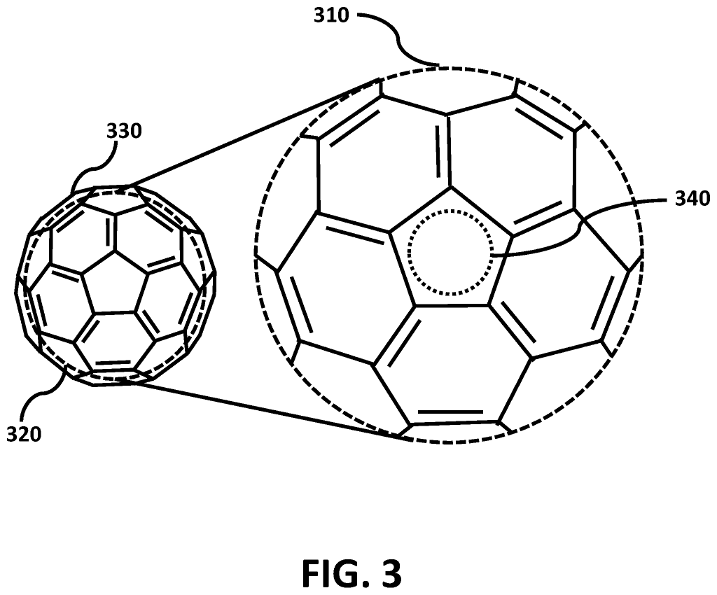

FIG. 3 is an illustration of an enlarged inset view 310 of one portion of a molecular structure of buckminsterfullerene molecular nanoparticle 330. The enlarged partial section 310 is a flat perspective of the encircled region 320 on the complete perspective view of C60 330. A dotted circular region 340 indicates a pentagonal carbon to carbon bonded structure which is highly strained and therefore is most reactive to chemical functionalization.

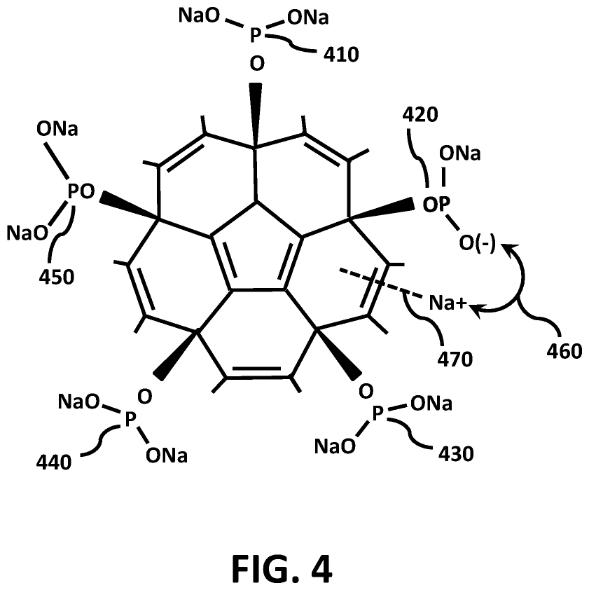

FIG. 4 is an illustration of one portion of a molecular structure of buckminsterfullerene sodium phosphonates provided with the reversible hopping pi-cation bonded sodium adducts of these teachings. Five covalently bonded phosphonate groups 410, 420, 430, 440 and 450 are deposed such that the distance between the sodium atom of at least one phosphonate group and the fullerene group is less than 5 nanometers. This leaves many carbon atoms in the molecular carbon cage available to accept hopping of a multiplicity of sodium cations 470 from the sodium phosphonate groups to form aromatic pi-cation bonds 470 to function as the nano-surfactant. The pi-cation bonded sodium ion is able to penetrate the symmetric charges that hold together the assembled proteins at the hydrophobic regions of viral protein structures such as capsid outer shells and to penetrate and disassemble the spike glycoproteins pinned by chloride ions in many types of viruses.

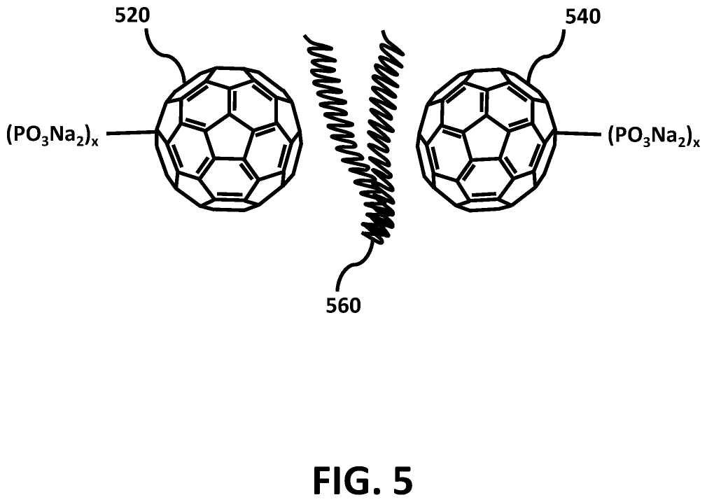

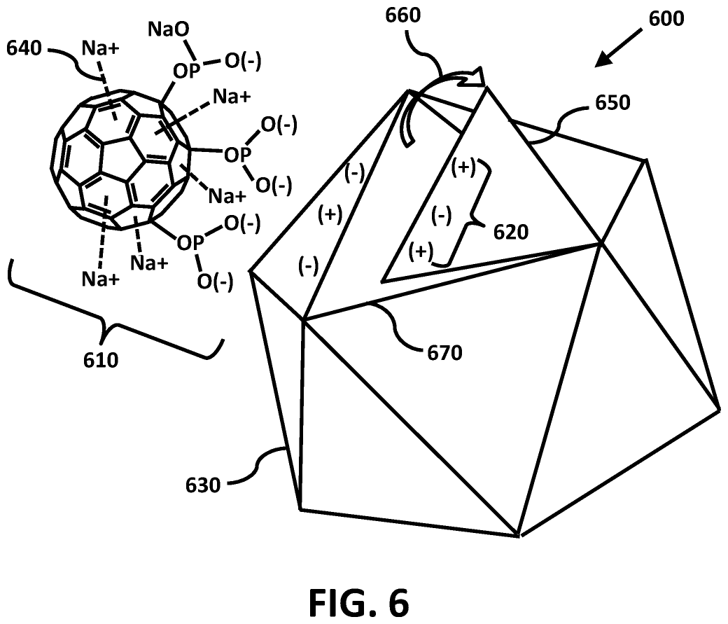

FIG. 5 is an illustration of the nano-surfactant chaperoning of a therapeutic molecule 560 such as an antibody protein, a strand of mRNA, or a drug. Transiently coupled by induced van der Waals charge attraction between the molecular structures of fullerene sodium phosphonates 520, 540 is a therapeutic molecule 560, for example, a poorly soluble antibody, mRNA, or drug that has proven efficacy in vitro but is otherwise unable to traverse the blood-brain-barrier, the digestive cell lining, or to become bioavailable for transport by the bloodstream and blood plasma. This therapeutic molecule 560 becomes coupled to the hydrophobic carbon faces of at least one fullerene sodium phosphonates 520, 540 by induced van-der-Waals forces. Simultaneously, the sodium phosphonate functional groups of the fullerene sodium phosphonate serve to form electrostatic faradic bonds with the hydrophilic biomolecules of pulmonary surfactant, thereby enabling their combination with blood plasma and expediting their transport throughout the body of the patient. These properties make coupling of at least one of the fullerene sodium phosphonates 520, 540 with the therapeutic molecule useful against various types of bacterial and fungal infections of the lung such as antibody-resistant bronchitis and tuberculosis, by enhancing the solubility, transport, and delivery of the therapeutic molecule especially by directly treating the affected lung tissues. While it is indicated that the desired therapeutic molecule 560 is sandwiched between two fullerene sodium phosphonates 520, 540, it is understood that for large, poorly soluble, and complex therapeutic molecules, transient coupling with any number of fullerene sodium phosphonates is acceptable. The FSP-drug complex can self-assemble more rapidly with the assistance of an applied electric potential during the self-assembly process to provide the requisite enhanced bioavailability for the delivery function, according to these teachings. FIG. 6 is an illustration of a van-der-Waals induced charge disruption of a viral capsid 600. Fullerene sodium phosphonate 610, because of its small size, penetrates the symmetric induced charges 620 holding together the assembled protein plates at the hydrophobic regions of the capsid outer shell of a virus particle 630. The hopping sodium cations are provided with a multiplicity of aromatic pi-cation bonds 640 functioning as the nano-surfactant to penetrate and disrupt the induced charges which hold together the edges of the capsid plates. This results in the disassembling of a first capsid molecular protein plate 650 as shown by the uplifting direction of the large, curved pointing white arrow 660 from the capsid assembly. The region of the opening 670 provides access to further solubilize and remove viral RNA from inside the viral particle capsid assembly 630. The process illustrated here applies to all types of viruses of which strains and types in any configuration or geometry whatsoever are included without limitation, according to these teachings.

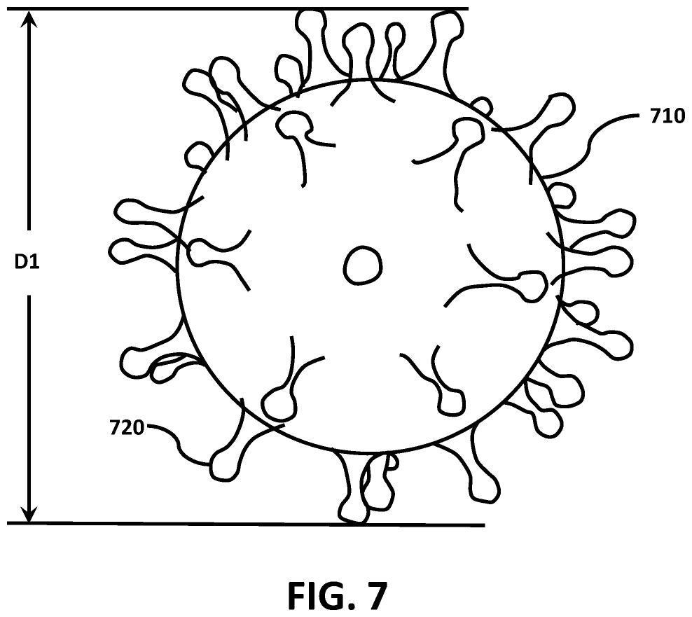

FIG. 7 is an illustration of an influenza or a corona virus having spike glycoproteins at the surface of a globular capsid that is suitable for disruption using the sodium phosphonate nanoparticle molecular structure. Virus particle 710 has a globular structured capsid that is typical for many types of enteric viruses such as influenza, and the novel corona viruses including that of SARS-COV-2 and its many variants. The size of this virus particle indicated by dimension D1 is about 250 nanometers. A multiplicity of coronal spike glycoprotein structures 720 emerge at the surface of the viral capsid and serve as receptors for target proteins that are used to signal the unravelling of the viral capsid for the purpose of combining the viral proteins and viral RNA within a suitable host cell by penetrating the cell membrane and injecting its contents, where this is the first step of the pathogenic viral invasion process and is better known as endocytosis.

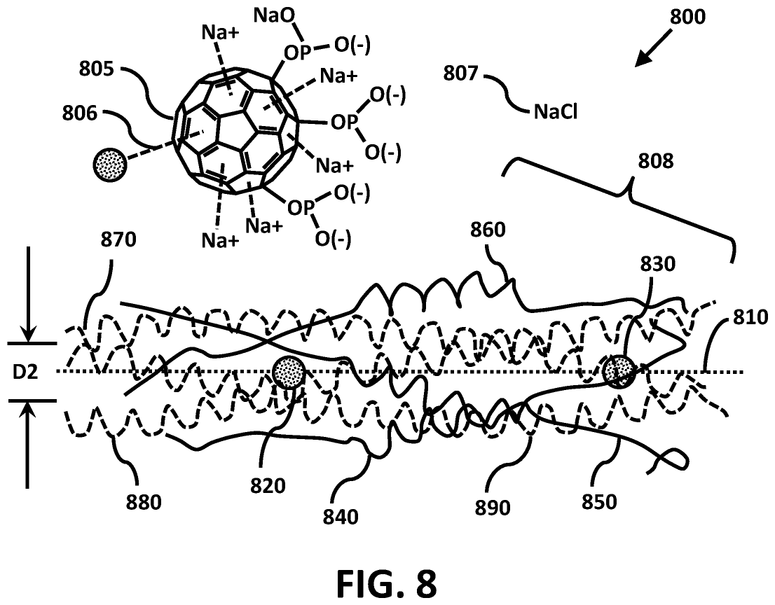

FIG. 8 is an illustration of a side view of the chloride ion stabilized viral spike glycoprotein of an exemplary influenza or novel coronavirus such as COVID-19 or SARS-Cov-2. The fullerene sodium phosphonate nano-surfactant must mediate the unzippering of this spike glycoprotein outside of any cell membrane to proactively avoid endocytosis. The mechanism to do this is to generate conditions sufficiently like endocytosis that the first step required to unravel the globular capsid protein shell to which it is attached can be performed outside of susceptible cells. Once opened and unraveled, macrophages and other aspects of the immune system can safely dispose of the viral protein fragments including the exposed viral RNA.

The fullerene sodium phosphonate 805 is not drawn to scale, however it must be of sufficiently small size to be capable of accessing the region where negatively charged chloride ions stabilize some types of virus particles or their structures to enable the formation of an aromatic pi-anion bond between the fullerene group and the chloride ion as indicated by a dashed line 806.

Chloride ions extracted in this manner are immediately exposed to a multiplicity of hopping sodium cations on the fullerene sodium phosphonate and may then leave the vicinity of this molecule as a sodium chloride salt (NaCl) 807. Extraction of chloride ions from the interior of the viral spike glycoprotein assembly 808 will cause the unravelling of this assembly as the charge balance of this molecular structure is disrupted. The viral spike glycoproteins are stabilized by a multiplicity of chloride ions 820, 830 centrally located to the spike along the region of a dotted line 810. These chloride ions are ejected from the central hydrophobic region of the viral spike via van-der-Waals mediated charge disruption by the sodium hopping mediated surfactant properties of fullerene sodium phosphonate. The viral spike of the exemplary novel coronaviruses are composed of 6 entwined proteins of two fundamental types being the human receptor 1 (HR1) proteins indicated by the coiled coils of 840, 850, 860 and the HR2 proteins indicated by the dashed coiled coils 870, 880, 890. Fullerene sodium phosphonate penetrates to the hollow tubular interior of these spike viral structures to disrupt the charge symmetry and therefore the stability of these structures, causing them to eject the stabilizing chloride ions at the interior of these molecular structures to thereby unravel the viral spike glycoproteins before they can locate a cell membrane to initiate endocytosis and become invasive to a cell of the body. This premature unravelling process is enabled by the fullerene sodium phosphonate, which has a size that is smaller than the size of the hollow central gallery region indicated by D2 which is enclosed by the spike glycoproteins.

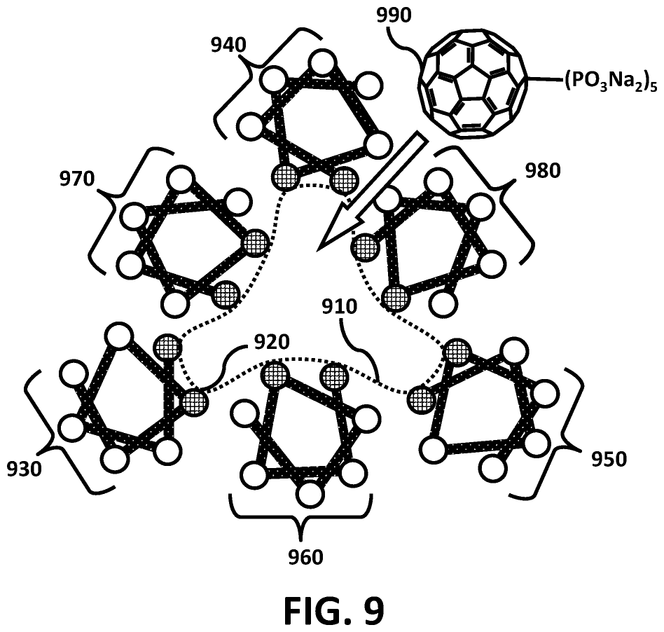

FIG. 9 is an illustration of a cross-section view of the viral spike glycoproteins illustrated in FIG. 8. The central hydrophobic hollow region of the coronavirus spike glycoprotein has an approximately triangular shape as indicated by the dotted line 910. Such structures are well documented in the x-ray crystallography literature for these viruses. The hydrophobic character of the interior region is determined by the presence of hydrophobic amino acid residues indicated by the multiplicity of circular crosshatched pattern circles 920 that abut the hydrophobic region. Viral spike glycoproteins HR1 are represented by the structures 930, 940, 950 and viral spike glycoproteins HR2 are represented by the structures 960, 970, 980 wherein each of these structures has hydrophilic amino acid residues indicated by the multiplicity of white circles at the outside regions which face the water-soluble intercellular environment in which the virus must travel to invade the cells of the body. The large white arrow shows the direction that fullerene sodium phosphonate 990 travels to enter the hydrophobic central region of the coils of the spike glycoproteins. The fullerene sodium phosphonate entry process is enabled by the nano-surfactant sodium cation-aromatic pi bond hopping mechanism.

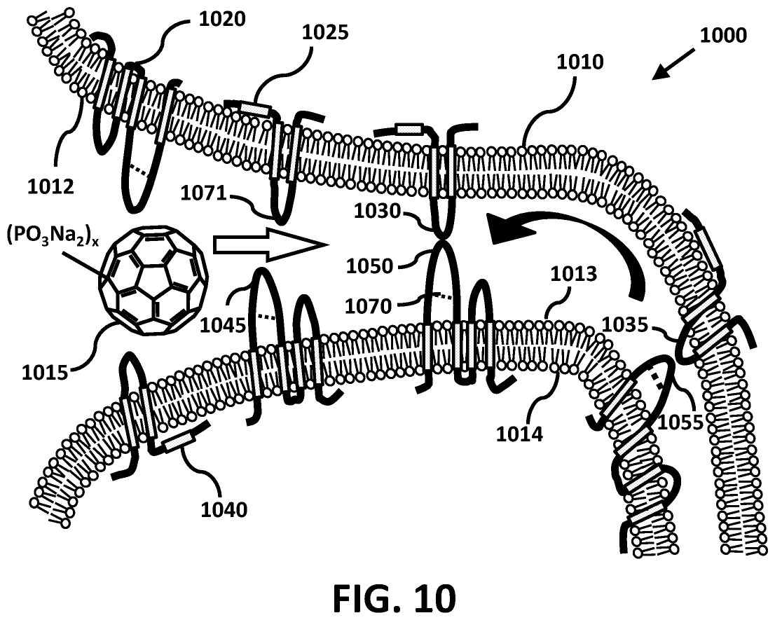

FIG. 10 is an illustration of the recovery process of zippered phospholipid cell membranes 1010, 1014 that have been usurped and repurposed as viral replication platforms. The region of the white arrow denotes an electrostatic bilayer in transition away from the zippered region of electrochemical charge storage between opposing charged van-der-Walls hydrophobic faradic viral protein loops in the nanoconfined spaces of the region of the black arrow. Fullerene sodium phosphonates 1015 enables the separation and restoration of cell membranes by being neither a purely electrostatic material or a purely faradaic material. It should rather be regarded as a catalyst that enables a continuous transition between the two types of regions determined by the extent of sodium ion solvation and hydrophobic van-der-Walls fullerene charge interaction. This is the region in which the pseudocapacitive processes are observed by the exchange of ionic sodium from the phosphonate groups to pi-cation type bonding with the polarized charges induced at the fullerene carbon face. This hopping of sodium ions from one region of chemical bonding to another region of chemical bonding at the nanoparticle molecular structure is not an ideal primary bonding type of exchange, such as from a covalent type to and ionic type of bond. The hopping of the sodium ions is a reversible process in which the exchange of locations takes place from a somewhat non-polarizable (faradaic) region at the phosphonate to a somewhat polarizable (non-faradaic) region at the fullerene carbon face over a hopping distance of less than five nanometers. The unzippering process constitutes a subtle chemical mechanism that has now been harnessed to reverse a pathological biochemical condition operating on infected cellular components as propagated by an invasive virus.

The external phospholipid membrane of the endoplasmic reticulum of a mitochondrion 1010, 1014 are facing the cellular cytoplasm, and internal phospholipid membranes 1012, 1013 are facing the endoplasmic reticulum lumen. The electrostatic zipper function of an invasive pathogenic virus such as an influenza or a corona virus is enabled by large luminal viral protein loops, having opposite induced van-der-Walls charges at the tips of the curvatures of these loops at opposing internal membrane regions. Such loops are termed ‘nonstructural integral membrane proteins’ or nsp; their operation is demonstrated by the coupling bonds of the abutting hydrophobic regions of high curvature in the dark black curved lines representing complementary nsp structures 1030, 1050, and complementary nsp structures 1035, 1055. Large, looped regions of each of the characteristic double loops of nsp4 structures 1020, 1045, 1050, 1055 facing the interior of the mitochondrion at the lumen are collectively represented by 1070 and are each stabilized by a sulfur-sulfur bridge bonding structure represented by the dotted line across large luminal loop 1070. Nsp3 luminal loop structures 1025, 1030, 1035, 1040 provide a complementary electrostatic and hydrophobic bond forming region at the point of high curvature of the single inward facing luminal loop, collectively represented by 1071. The action of nsp3 to nsp4 hydrophobic electrostatic bonding bridges these opposing luminal loops of type 1030 to type 1050, providing a zippering attractive force to bring opposing inner walls of the mitochondrion into local proximity and hold them together. The narrowed gap region between the now proximal phospholipid membranes serves as a platform to assemble the replicating virus. The zippering direction of this movement is shown by the large curved black arrow. Fullerene phosphonate 1015 is provided to counteract the loop zippering function, by inserting itself into the regions between abutting nsp3 1071 and nsp4 1045, as indicated by the pointing direction of the large white arrow. This allows electrostatic charges to be introduced to at least one hydrophobic portion of 1045 and 1071 by the induced bonding of a hydrophobic portion of the fullerene sodium phosphonate 1015.

It is notable that the physiological pH within the mitochondrion will cause negative ionic faradic charges to appear at the terminal ends of at least some of the pendant phosphonate groups. Faradic charge is the expression of continuous electrostatic interactions that exist between charged or polar surfaces and extend into water which is a polar molecule. However, transient or induced charges appear at the fullerene group, where the carbon faces provide induced van der Waals forces to create a transient opposing charge in an uncharged or non-polar hydrophobic abutting surface, such as provided by the ability to adhere to a first nonpolar nsp viral protein. Those portions of the molecular structure provided with charged fullerene phosphonates being of hydrophilic or polar nature are then able to repel the hydrophobic region at the point of maximum curvature of any second abutting or nearly abutting nsp loop. The fullerene phosphonate thereby acts to electrostatically cap to prevent the induction of opposing charges with any zippering nsp luminal loop. This unzippering function of the fullerene phosphonates is designed to disable the nsp from finding and recruiting any partner nsp loop for the purpose of establishing the hydrophobic zipper of the platform required to replicate virus particles. Similar paired nsp types of viral protein structures to those of coronavirus nsp3 and nsp4 have been identified in hepatitis virus, especially that of hepatitis B or (HBV). It is therefore the purpose of the present invention to halt the recruitment of partnering nsp of any type, from any virus particle proteins expressing a van der Waals paired nsp zipper function. The electrostatic or faradic capping function of C60 fullerene phosphonates halts the replication of virus from creating replication platforms within cellular mitochondria or other cellular organelles when the fullerene sodium phosphonates promote conditions unfavorable to viral replication, according to these teachings.

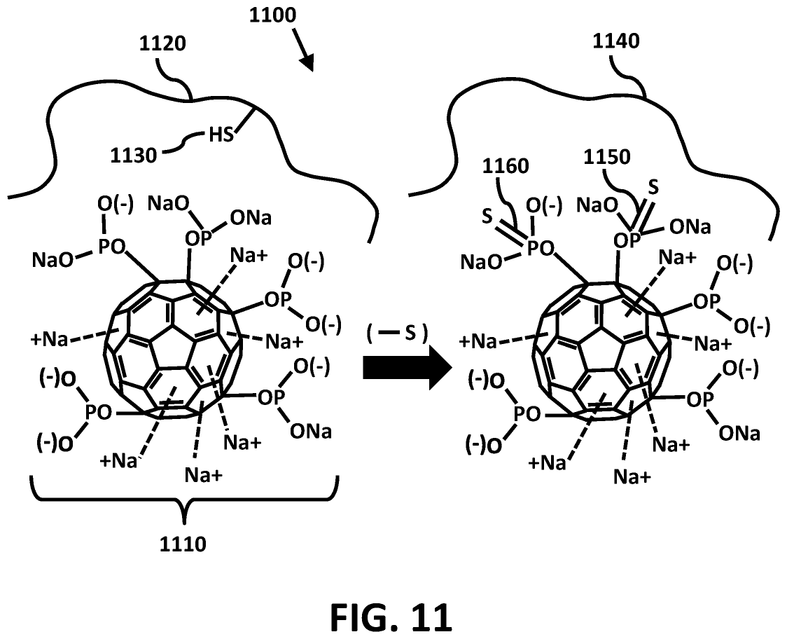

FIG. 11 is an illustration of the desulfurization reaction between sodium phosphonates to extract catalytic sulfur from a cysteine or other sulfur containing amino acid from within the protected cavity of a viral or fungal protease. The desulfurization reaction proceeds in the direction of the large black arrow indicating (−S) for the sulfur extraction. The size of the fullerene sodium phosphonate 1110 must be sufficiently small to enable entry into the protected protease catalytic fold or cavity within the overall protease structure 1120. The catalytic sulfur or sulfhydryl group at a cysteine or other sulfur containing amino acid 1130 resides within a protected cavity of the viral or fungal protease. The extraction of sulfur from the protease 1140 leaves this protease unable to digest the proteins of the human body and thus renders it unable to provide raw materials in the form of amino acids to convey to the invasive fungus or viral replication platform. Some of the unreacted sodium phosphonates exposed to a sulfur containing compound in this case may remain in an oxidation state of 3 and are yet unreacted with sulfur. However, at least one abutting phosphorous atom reacts to form a sulphurated phosphate 1150, 1160 which is now at an oxidation state of 5 in this example of the desulfurization reaction. Desulfurization assists the human body to penetrate and deactivate destructive viral and fungal proteases, such as the main protease 3 (MPRO3) associated with the SARS-Cov-2 coronavirus strains.

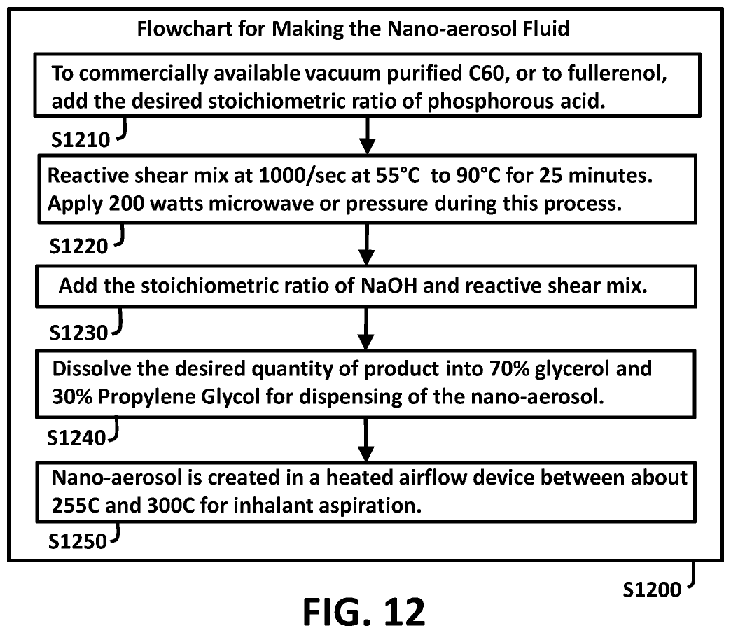

FIG. 12 is a flowchart representation of an exemplary scalable method S1200 for the synthesis of a nano-aerosol formulated for inhalant administration of fullerene sodium phosphonate (FSP). In step S1210 one mole of commercially available vacuum purified C60, or fullerenol, is combined with a desired stoichiometric ratio of dry crystalline phosphorous acid. It is noted that the use of stoichiometry for fullerenol starting material is to be adjusted based on the number of poly-hydroxylations of the fullerenol for the purpose of one type of chemical reaction for making fullerene sodium phosphonates. In some embodiments there are no residual, impurity, or excess polyhydroxylated fullerene remaining after the use of this method to compromise the purity of the resulting fullerene sodium phosphonate formulation. In step S1220 the prepared dry powder mixture is reaction shear milled in a temperature range from 50° C. to 95° C. at 1000 per minute shearing rate to achieve the desired product fullerene phosphonate. In this process, a shear pressure of about 20 grams per square micron is sufficient to create a slightly geometric oblate spheroid of the C60 functional group to shift the density of states of the electrons of the carbon cage molecule into transient anisotropic electrostatic charge distributions suitable for reaction. Alternatively, microwave irradiation will be able to induce the required electrostatic dipoles for this reaction to proceed. In step S1230, a stoichiometric amount of sodium hydroxide is added to the produced fullerene phosphonate and the reactive shear mixing process is continued for another 25 minutes. In step S1240, a desired concentration of product is created by dissolving a weighed amount of the dry fullerene sodium phosphonate powder into a solvent mixture such as 70% glycerol and 30% polypropylene glycol by volume mixture. In step S1250, a metered amount of the nano aerosol fluid from step S1240 is generated, for instance, by a commercially available electronic dispensing device suitable for client inhalant aspiration by means of a heated airflow between about 255° C. and 300° C. to create the nano-aerosol, according to these teachings.

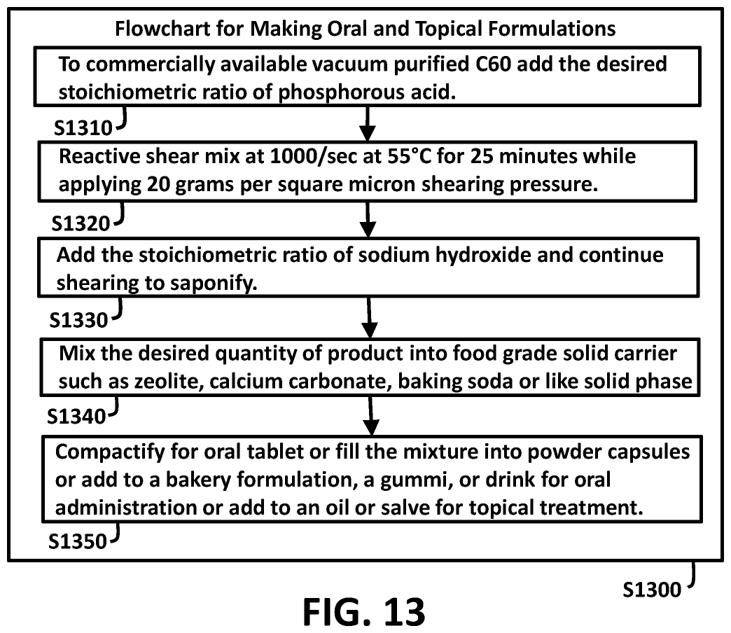

FIG. 13 is a flowchart representation of an exemplary scalable method S1300 for making formulations of fullerene sodium phosphonates for various exemplary administration methods. In step S1310 one mole of commercially available vacuum purified C60 is combined with a desired stoichiometric ratio of phosphorous acid. In step S1320 the prepared dry powder mixture is reaction shear milled in a temperature range from 50° C. to 95° C. at 1000 per minute shearing rate to achieve the desired product fullerene phosphonate. In this process, a shear pressure of about 20 grams per square micron is sufficient to create a slightly geometric oblate spheroid of the C60 functional group to shift the density of states of the electrons of the carbon cage molecule into transient anisotropic electrostatic charge distributions suitable for the reaction. In step S1330 a stoichiometric amount of sodium hydroxide is added to the produced fullerene phosphonate. The reactive shear mixing process is continued for another 25 minutes to incorporate sodium in the saponification reaction. In step S1340 the desired quantity of product is mixed into a food grade solid carrier such as a zeolite, baking powder (sodium carbonate), calcium carbonate, baking soda (sodium bicarbonate), collagen powder, natural or artificial sweetener, or similar solid phase to produce an edible product. For example, an exemplary 1 kilogram loaf of antimicrobial bread can contain 10 grams of baking soda or baking powder fortified with 500 mg of fullerene sodium phosphonate to yield 50 mg of fullerene sodium phosphonate per slice of bread at 10 equal slices per loaf, such that a daily administration of two slices provides 100 mg fullerene sodium phosphonate per day.

In optional step S1350 the selected edible solid phase is compacted to produce an oral tablet, or the mixture is added into hard gelatin powder capsules for exact dosages or added to a bakery formulation to create a pastry or cookie or processed with gelatin and heating to make an edible gummi. For topical formulations, the fullerene sodium phosphonate is added to an oil such as avocado oil and a waxy petrolatum such as petroleum jelly to make a topical lotion and salve applied by rubbing onto the affected skin tissues to treat antibiotic resistant skin fungus or antibiotic resistant MRSA skin infections or other types of microbial skin infections. It is understood that other methods for oral consumption and administration or variations of these methods can be found satisfactory and able to convey an amount of fullerene sodium phosphonates to the human or animal patient.

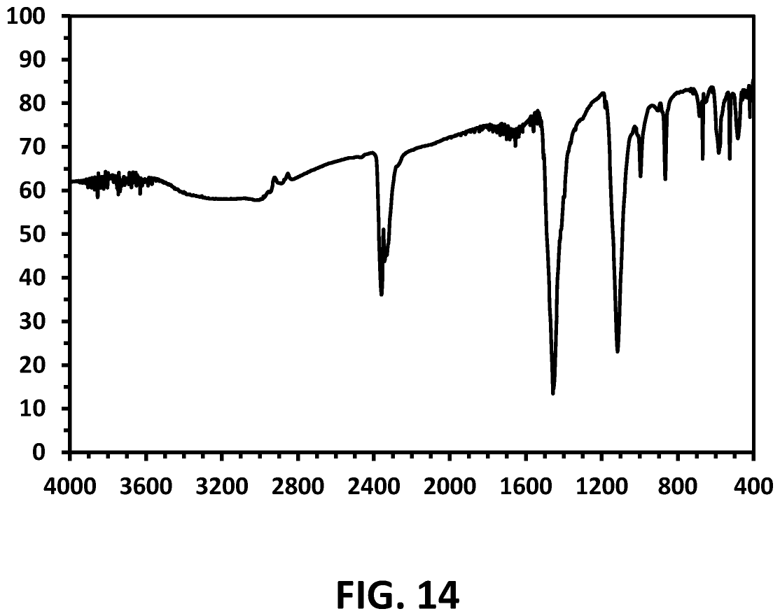

FIG. 14 shows experimental FTIR data for fullerene sodium phosphonate. This sample tested by FTIR was prepared by a method of mixing, crushing, and consolidating under 7 metric tons of pressure, about 0.001 grams of analyte with 1 gram of a diluent solid material that is substantially transparent to infrared light, the diluent here being anhydrous potassium bromide (KBr), which then flows under pressure to form a translucent pellet of about 0.4 mm thickness. Spectral background subtraction in air using a pure KBr control pellet of the same mass and thickness was used to obtain a baseline instrument infrared spectral transmission response. This method is generally referred to as the ‘KBr pellet’ sample preparation method, and it is used hereinafter throughout for each FTIR experimental data collection and spectral analysis. The Fourier transform infrared spectrophotometer used herein to obtain the FTIR spectrum is a model RF6000 FTIR instrument manufactured by Shimadzu of Japan.

The weak absorbance from 2900 cm⁻¹ to 3500 cm⁻¹ indicates few hydroxyl groups are present in this sample, indicating a good completion of the saponification reaction as the sodium has well neutralized the acidic phosphonate groups. The absorbance peak at 2359 cm⁻¹ can be ignored as this arises from local variations in the atmosphere of carbon dioxide in the laboratory where the test was performed. A strong and sharp absorbance peak at 1456 cm⁻¹ is attributed to the presence of the phosphonyl (P═O) group. Another strong and sharp absorbance peak at 1117 cm⁻¹ is attributed to the presence of the oxy-sodium (O—Na) stretching vibration seen in saponified molecular structures. The medium intensity and very narrow peak at 526 cm⁻¹ is attributed to the fullerene carbon aromatic resonance vibration that is normally accompanied by a 726 cm⁻¹ absorbance mode, however this second mode now appears at 669 cm⁻¹ wherein the shift in this mode is attributed to the presence of aromatic pi-cation sodium (Na+) bonding interactions.

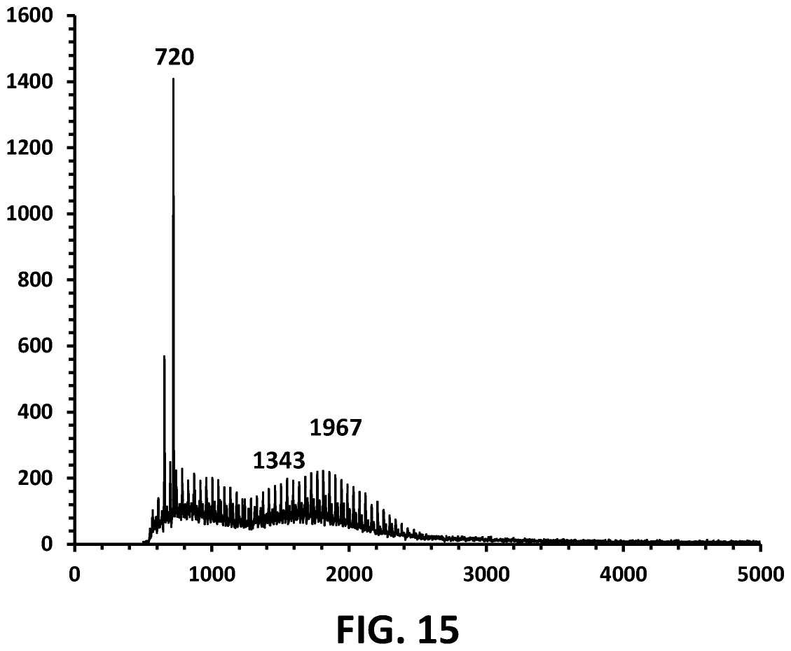

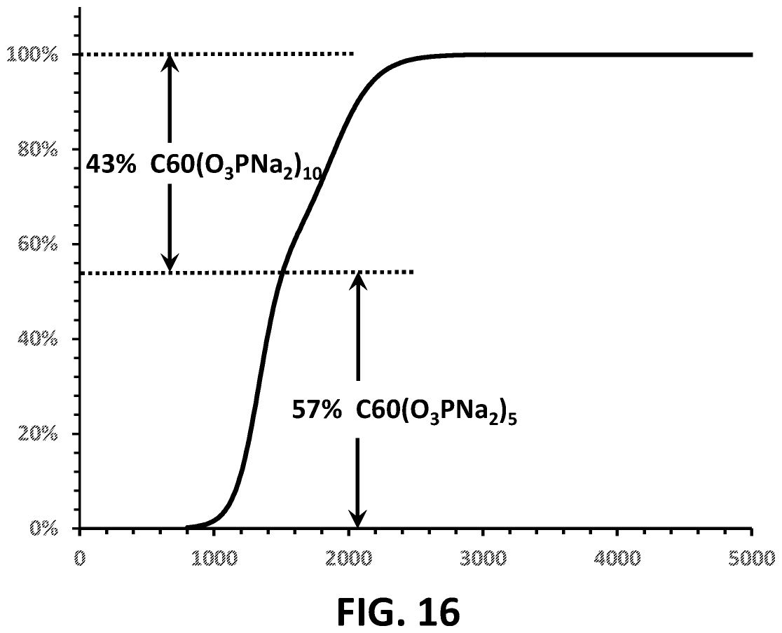

FIG. 15 shows experimental negative mode MALDI-TOF mass spectrograph data for the five and ten sodium phosphonate group derivatives of C60. This test sample, and each of the MALDI-TOF test data that follow, were prepared by dilution with acetonitrile, after which the aqueous sample was introduced in a vaporized state into a Voyager Mass Spectrograph from Applied Biosystems (Foster City, Calif., USA). Negative mode bombardment, resulting in ion formation, was by fast moving electrons at about 70 eV energy. One electron from the highest orbital energy was dislodged, and therefore, molecular ions were formed. Some of these molecular ions underwent spallation, and subsequent fragment ions were formed. The fragmentation of the ions occurs because of laser energy that is applied to the sample within the ionization chamber. Only negative ions were recorded. The largest peak observed was the primary and core molecular ion, this being a fullerene ion as indicated by the numeric peak label at mass to charge ratio of 720. The primary molecular ion was subsequently verified using a pristine pure reference material of C60 tested immediately after this test, under both negative mode and positive mode test conditions (the check standard results are not shown here). The observed mass chromatographic spallation ions greater than the primary molecular ion, formed peaks that were separated into two distinctly charged groups with their respective peak masses clustered about local maxima at 1343 and 1967 mass to charge ratios (m/z), and were respectively assigned to a nominal C60(O₃PNa₂)₅ composition, and a trace nominal C60(O₃PNa₂)₁₀ composition based on a combinatorial ionic mass and charge analysis. This interesting result is interpreted to indicate that phosphonates substantially bond at the highly strained pentagonal carbon structures of the fullerenes; the pentagonal regions have five carbons at their vertices, and these five carbons appear to have the most reactivity compared to those carbon atoms occupying the vertices of the less strained hexagonal facets. This hypothesis was tested in a sample prepared to react with more phosphonates in FIG. 16. It is understood, however, that fullerenols have random numbers and positions of hydroxyl groups, therefore a preferred phosphonate addition sequence does not apply to the case of making a fullerene sodium phosphonate sample using a fullerenol starting material.

FIG. 16 is an illustration of the integrated peak area function for the data of FIG. 15. The analysis to determine the composition is as follows. The atomic mass weight of each atom in the product is known because the reactants consist of carbon of nominal mass 12, oxygen of nominal mass 16, hydrogen of nominal mass 1, phosphorus of nominal mass 31, and sodium of nominal mass 23. The functional group disodium phosphonate is (—O₃PNa₂), and the weight sum of each atom in this spallation product is three oxygen plus one phosphorus plus two sodium, or 125 atomic mass units. When one of these spalls from the fullerene core molecule, a fragment remains that weighs 125 less than the starting material. In this way it is possible to add the weights of such fragments together to arrive at the composition for the group peak weight at the indicated charge as measured by the detector, being designated as an observed mass divided by the observed charge. First, the raw data was linearly baseline corrected, and the sum of each of the peaks was then integrated. The integrated peaks were then normalized to the maximum value of all peaks integrated, to arrive at 100% of all charged spallation chromatographic peaks greater than the core fullerene. This data was then imported to Jandel Scientific (San Rafael, Calif. USA) commercial mathematical curve fitting Tablecurve2d software, used for spectroscopy, chromatography, and electrophoresis. A user defined additive sum of two sigmoidal functions was used to fit this data. The resulting respective fitted sigmoidal transition function centers corresponded well with the two maximum peak values of their respective cluster of peaks in the source experimental data, and the sum of both sigmoidal functions equaled unity at 100 percent. The sigmoidal amplitude of 0.57 corresponds with the 57% composition associated with nominal C60(O₃PNa₂)₅ composition, and the sigmoidal amplitude of 0.43 corresponds with the nominal 43% C60(O₃PNa₂)₁₀ composition. Additional corroborating verification was achieved and confirmed using this type of analysis for the same tested material composition under positive mode MALDI-TOF (not shown).

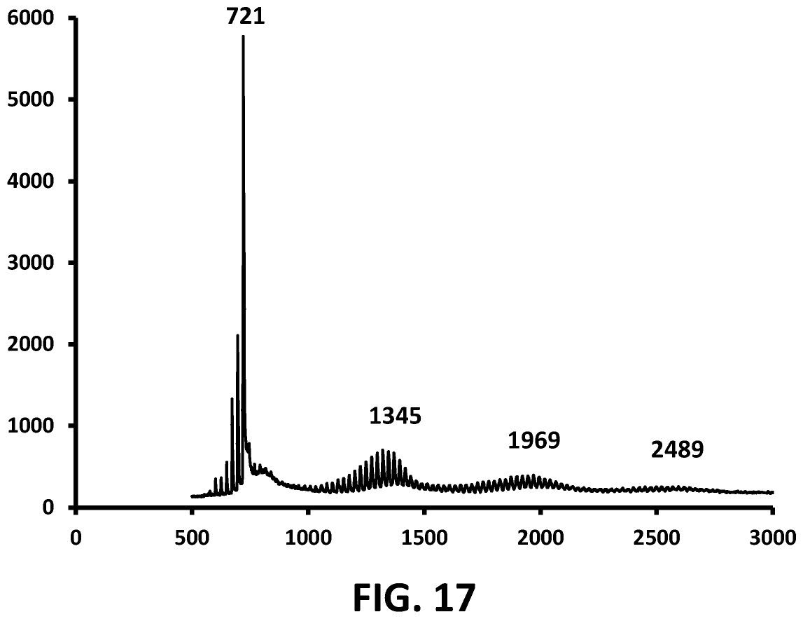

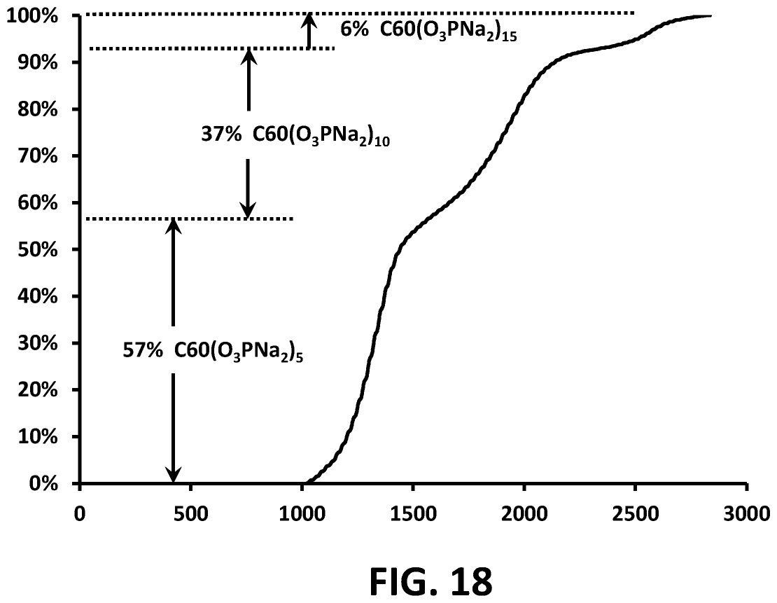

FIG. 17 is an illustration of the experimental negative mode MALDI-TOF mass spectrograph data for five, ten, and fifteen sodium phosphonate group derivatives of C60. The observed mass chromatographic spallation ions, greater than the primary molecular ion, formed peaks that were separated into three distinctly charged groups with their respective peak masses clustered about local maxima at 1345, 1969, and 2489 mass-to-charge ratios, and were respectively assigned to a nominal C60(03PNa₂)₅ composition, a nominal C60(O₃PNa₂)₁₀ composition, and a nominal C60(O₃PNa₂)₁₅ composition based on a combinatorial ionic mass analysis. This test sample is interpreted to have a different distribution of 5, 10, and 15 phosphonates as compared to the prior sample of FIG. 15, because a greater quantity of phosphonates was added, where this interpretation is based on the analysis result for FIG. 18.

FIG. 18 is an illustration of the integrated peak area function for the data of FIG. 17. The analysis tools, software, and procedure used to construct this integration analysis are as described for FIG. 16. A user defined additive sum of three sigmoidal functions was used to fit this data from mass to charge ratio of about 1020 to a cutoff value of mass to charge ratio of about 2832, above which no chromatographic peaks were detected. The resulting respective fitted sigmoidal transition function centers corresponded well with the three maximum peak values of their respective cluster of peaks in the source experimental data, and the sum of all sigmoidal functions was normalized to equal unity or 100 percent. The sigmoidal amplitude of about 0.57 corresponds with the nominal C60(O₃PNa₂)₅ composition, the sigmoidal amplitude of about 0.37 corresponds with the nominal 37% C60(O₃PNa₂)₁₀ composition, and the sigmoidal amplitude of 0.06 corresponds with the nominal 6% C60(O₃PNa₂)₁₅ composition, wherein the latter about 6% of product is associated with the slight excess of phosphorous acid deliberately used to indicate an ability to perform synthesis of substituent phosphonate groups greater than 10 for the purposes of this demonstration. Additional corroborating verification was achieved and confirmed using this type of analysis for the same tested material composition under negative mode MALDI-TOF (not shown). Therefore, the hypothesis proposed for the preferential addition of phosphorous acid to fullerene to react as groups of five at the pentagonal regions shows experimental support for the structural interpretation being substantial addition as five groups of phosphonates. It is understood, however, that fullerenols have random numbers and positions of hydroxyl groups, therefore a preferred phosphonate addition sequence does not apply to the case of making a fullerene sodium phosphonate sample using a fullerenol starting material.

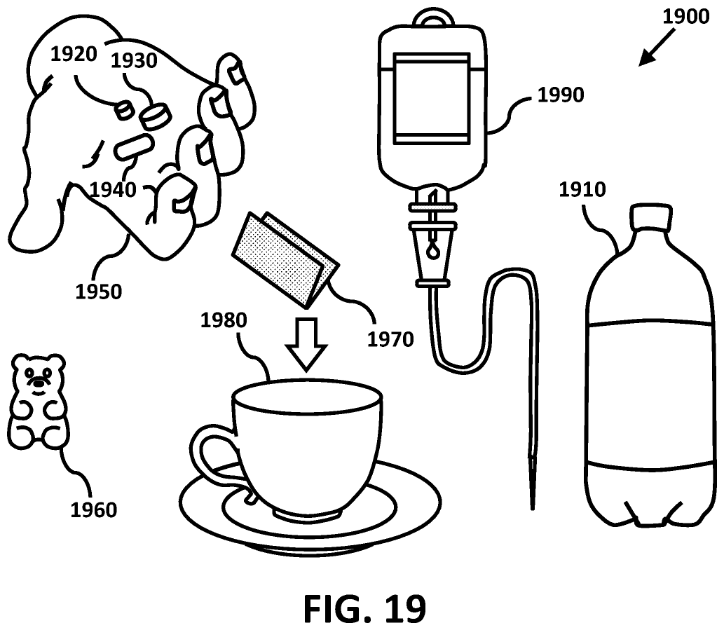

FIG. 19 is an illustration of exemplary methods to administer formulations to contain and deliver fullerene sodium phosphonates. In these non-limiting examples, the fullerene sodium phosphonate is dissolved into a carbonated soft drink, optionally including an alcohol content. It is also understood that other types of beverages such as wine, champagne, beer, and fruit juices are amenable to the addition of fullerene sodium phosphonates. Another method of delivery or dosage of fullerene sodium phosphonate is by pill 1920, tablet 1930 or powder-filled hard gelatin capsule 1940 which are portable and easily administered by hand 1950. Dilution of fullerene sodium phosphonate into edible powders includes baking powder (sodium carbonate), and baking soda (sodium bicarbonate) in any combination. In an alternative delivery method, the fullerene sodium phosphonate can be added to a gelatin gummi having a soft pliable dry form that may be shaped as desired, such as in the form of a ‘gummi bear’ 1960. In yet another method of delivery or serving, the fullerene sodium phosphonate powder may be added to a sugar or a sugar substitute and packaged into individual sweetening packets 1970. Such packets can be opened and added to a liquid brewed beverage such as a tea or a coffee 1980. Another method of delivering fullerene sodium phosphonates is by intravenous infusion therapy (IV drip) 1990, in which the fullerene sodium phosphonate molecules are attached by faradic attraction to the biomolecules of human blood plasma for subcutaneous application; an infusion pump can be used to adjust the delivery rate through a catheter that is to be inserted into the localized area to be treated.

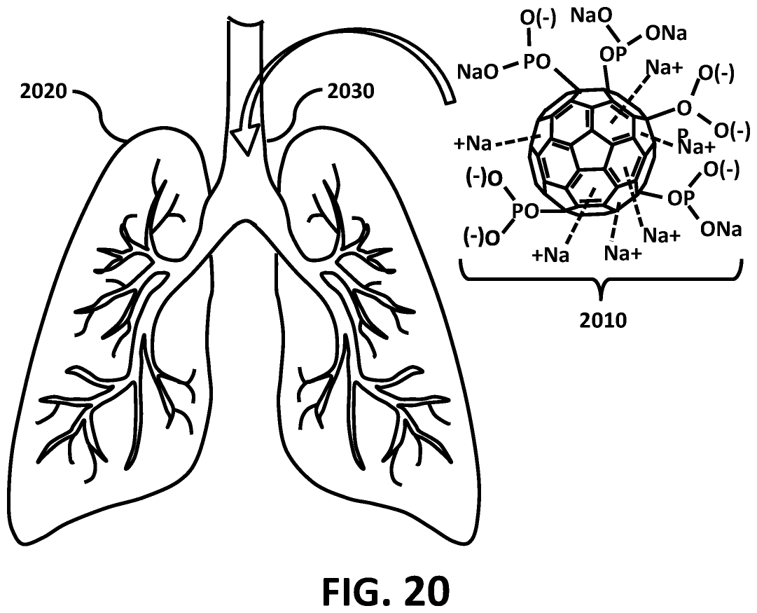

FIG. 20 is an illustration of fullerene sodium phosphonate aspirated into the lungs for preventative or therapeutic treatment of any microbial-induced respiratory ailment that can benefit from this method of breath mediated delivery. Fullerene sodium phosphonates 2010 are aspirated into the lungs 2020 and airways 2030 with a viral pneumonia or other invasive pathogen such as a fungal spore infection that causes a cytokine storm and filling of the lungs with mucus composed of pulmonary surfactant. Fullerene sodium phosphonates, being heavily negatively charged, is attracted to and binds with endogenously produced pulmonary surfactant (pus) in the airways. The surfactant properties of fullerene sodium phosphonate act to significantly reduce surface free energy, increase wetting ability, and lessen the viscosity of the biological mucus so that these biomaterials can be expeditiously returned as part of the blood plasma to the vascular circulatory system. The effect of the fullerene sodium phosphonate is to clear the lungs and airways of the patent so that breathing is no longer obstructed, and an exchange of oxygen and carbon dioxide gases allows normal and healthy respiration to be established. The antifungal properties assist with the treatment of valley fever, as well as other respiratory illnesses such as pneumonia, chronic obstructive pulmonary disorder (COPD), and some types of bacterial infections of the lung such as antibody-resistant bronchitis and tuberculosis by directly treating the affected lung tissues, especially when the antibody or therapeutic drug molecule is delivered between fullerene sodium phosphonates, according to these teachings.

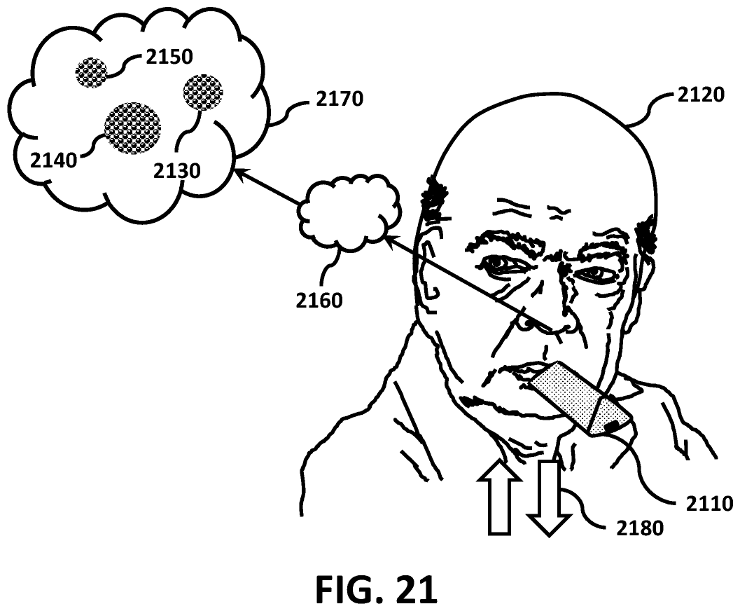

FIG. 21 is an illustration of the personal administration of an aspirated nano-aerosol delivery solution containing the fullerene sodium phosphonate molecules of the present invention. A nano-aerosol generating device 2110 filled with the fluid mixture containing the molecules of the present invention as an inhalant dispensing solution is provided for dispersing the created inhalant gas wherein the nano-particles are nebulized. The dispensing device 2110 may also be more commonly known as a nebulizer, or an electronic vaporizing device, or an electronic cigarette, or the functional part of a hookah to be shared among several users. In all cases these systems serve to carry the composition in a carrier fluid dispenser 2110, move that composition in nebulized form along with an aerosolized solvent, and transfer this composition into a substantially gaseous dispersion directed into the nose, mouth, trachea, and airways of a patient or user 2120. One intended use of the composition is to treat, delay or arrest the incidence of brain cancers wherein the nano-aerosol can expedite targeted delivery to the brain by avoiding a passage through the digestive system. Another intended use is to treat COPD, pneumonia, and other respiratory ailments using the composition of the present invention. A volume of an inhalant in the form of an air dispersed nano aerosol, a water mist dispersed vapor, an air dispersed powder, an air dispersed dust, or an air dispersed aerosol can be administered, in various embodiments. An exemplary daily dosing comprises an aspirated volume of 400 ml that is inhaled for 3 seconds for a cumulative dose of 0.0017 mg of fullerene sodium phosphonate delivered to the lungs and airway passages, dispensed from a solution or dust containing 500 parts per million fullerene sodium phosphonate.

Some of the nano-aerosolized composition is exhaled and shown as particulate clusters 2130, 2140, 2150 within exhaled smoke puffs 2160 and 2170 emitted on exhalation as indicated by the direction of thin line arrows pointing away from the nose of the subject 2120. Delivery of the nano-aerosol composition from dispenser 2110 provides antimicrobial properties to the mucus airway tissues wherein destruction of microbes associated with viral infection, fungal infection such as valley fever, or to treat COPD can be provided using this method. Systems that may be used for the method of dispersion of the nano-aerosol fluid is represented by dispenser 2110, and include, without limitation, any of the electronic cigarette devices produced internationally and listed in Appendix 4.1, “Major E-cigarette Manufacturers” of the “2016 Surgeon General's Report: E-Cigarette Use Among Youth and Young Adults” published by the Center for Disease Control and Prevention (CDC), Office of Smoking and Health (OSH) available at the CDC.GOV website, and/or any combination of piezoelectric, resistively heated, or inductively heated vaporized fluid delivery methods that can be utilized to deliver the composition of the present invention, especially when such a device is approved as a medical drug delivery device. Each embodied variation of such methods without limit are intended to aspirate aerosols as the method of therapeutic substance delivery of the composition of the present invention directed into the nasal cavities, mouth, tracheal breathing orifice, or intubated trachea of a patient. The supply direction of nebulized feed on inhalation and exhalation are delivered into the airways and lungs of the intended patient by the flow of supplied air as indicated by the direction of upward and downward facing large white arrows 2180, when used according to these teachings.

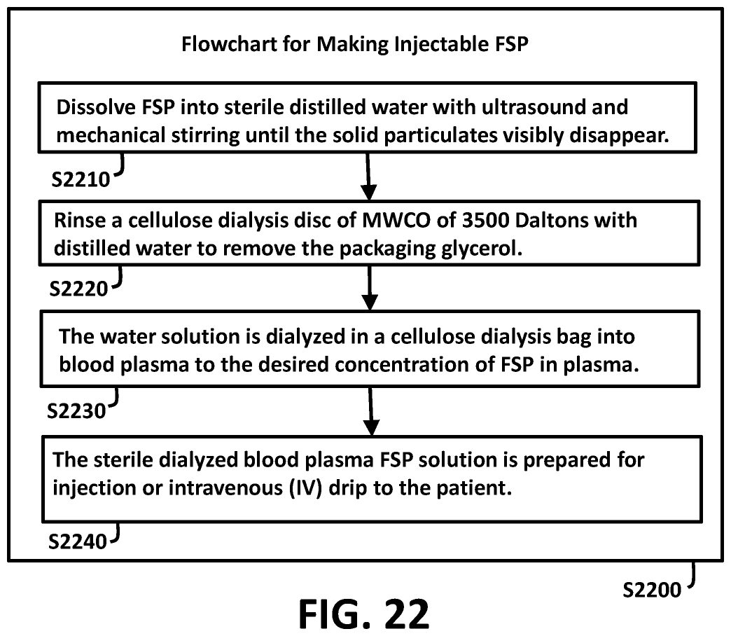

FIG. 22 is a flowchart showing exemplary steps to prepare fullerene sodium phosphonate in blood plasma for intravenous medical injection. In step S2210, fullerene sodium phosphonate (FSP) is dissolved into sterile distilled water with ultrasound and mechanical stirring until the solid particulates visibly disappear. In step S2220, a cellulose dialysis disc having a molecular weight cutoff of 3500 Daltons, for example, is rinsed with distilled water to remove any packaging glycerol. An exemplary 33 mm disc is the Spectra/Por™ RC, manufacturer number 132488, provided by Repligen at 18617 South Broadwick Street, Rancho Dominguez, Calif. 90220, USA. In step S2230, an aqueous fullerene phosphonate solution is dialyzed in a cellulose dialysis bag into blood plasma to obtain the desired concentration of fullerene sodium phosphonate in sterile blood plasma at physiological pH. By passing through the dialysis filter, potential impurities greater than 3500 Daltons as well as possible dust are removed. In step S2240, the sterile dialyzed blood plasma FSP solution is prepared for injection or intravenous (IV) drip to the patient at the rate specified by an attending physician. The drip rate may vary depending on the prescribed dosage and concentration, which can relate to the body weight of the patient as well as the severity of the microbial load to be addressed, which has been determined in the patient.

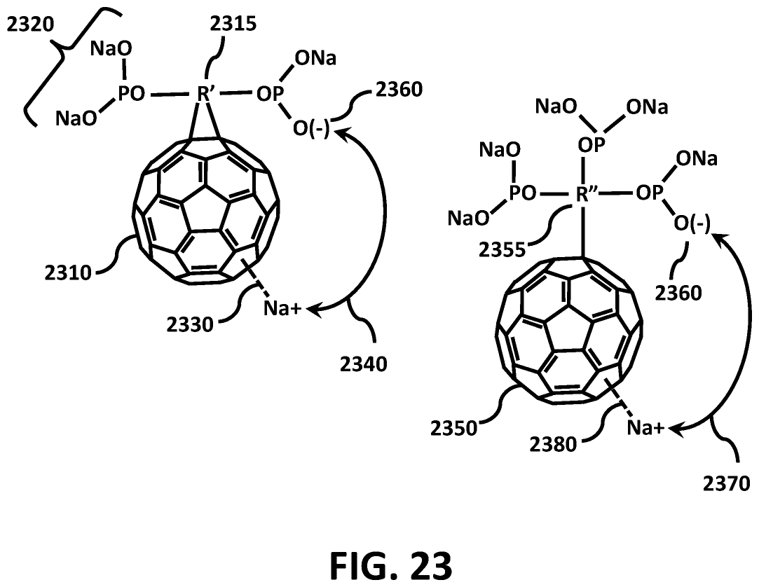

FIG. 23 is an illustration of alternative fullerene molecular structures with intermediate functional groups that contain sodium phosphonates having sodium ions that are capable of hopping to the fullerene. Bisphosphonate 2310 has an organic functional group containing at least one carbon that is bonded to two adjacent fullerene carbons represented by R′ 2315. The R′ group is bonded to two sodium phosphonates, where one of these is indicated by the bracketed region 2320. At least one sodium phosphonate 2330 is sufficiently proximal to the fullerene to permit reversible hopping to the state of pi-cation bonding to fullerene indicated by a dashed line in this molecular structure. More than one R-group 2315 with bisphosphonates 2320 may be bonded to the fullerene molecule. The overall size of fullerene bisphosphonates 2310 is sufficiently small and labile to allow insertion of at least one pi-bonded sodium cation at a hydrophobic carbon face to combine with a chloride pinning ion holding together a virus type to form NaCl and therefore destabilize and disassemble that viral capsid on the exit of the charge-stabilizing chloride ion.

Trisphosphonate 2350 has an organic functional group containing at least one carbon that is bonded to one adjacent fullerene carbon atom that is represented by R″ 2355. The R″-group is bonded to three sodium phosphonates. At least one sodium atom from a tris sodium phosphonate group 2360 is sufficiently proximal at less than five nanometers to permit reversible hopping 2370 to the state of pi-cation bonding to fullerene indicated by a dashed line 2380. More than one r-group 2355 with sodium tris-phosphonates 2320 may be bonded to the fullerene molecule. The overall size of fullerene tris-phosphonates 2310 is sufficiently small and labile to allow insertion of at least one pi-bonded sodium cation at a hydrophobic carbon face to combine with a chloride pinning ion holding together a virus to form NaCl and therefore destabilize and disassemble that viral structure on the exit of the charge-stabilizing chloride ion.

It is generally understood that the product of two simultaneous addition reactions onto the same fullerene molecule results in a bis-fullerene product, and that the product of three simultaneous addition reactions onto the same fullerene molecule results in a tris-fullerene product. Therefore, a single organic molecule adding to two carbon atoms of one fullerene is considered a bis-fullerene as for R′ 2315, whereas two of organic molecules of R″ 2355 adding to two carbon atoms of one fullerene is also considered a bis-fullerene. Three simultaneous addition reactions of either R′ or R″ to one fullerene molecule are generally considered tris-fullerenes even though three of the R′ may react with six carbon atoms of the same fullerene molecule. In all cases, there must be at least one phosphonate group on at least one reactant adduct to allow the subsequent reaction with sodium hydroxide to create a saponified bis-FSP or tris-FSP.

The antimicrobial properties of bisphosphonate fullerenes and tris-phosphonate fullerenes, being saponified phosphonates having an intermediate organic group that is bonded to the fullerene can be acceptable antimicrobial alternatives to fullerene sodium phosphonates having phosphorus directly bonded to the fullerene. It is to be understood that the pharmacokinetics and efficacious dosage can be different in such alternative structures, wherein it is generally known that greater mass molecular structures will diffuse more slowly than lighter molecular structures, in accordance with the teachings of the present invention.

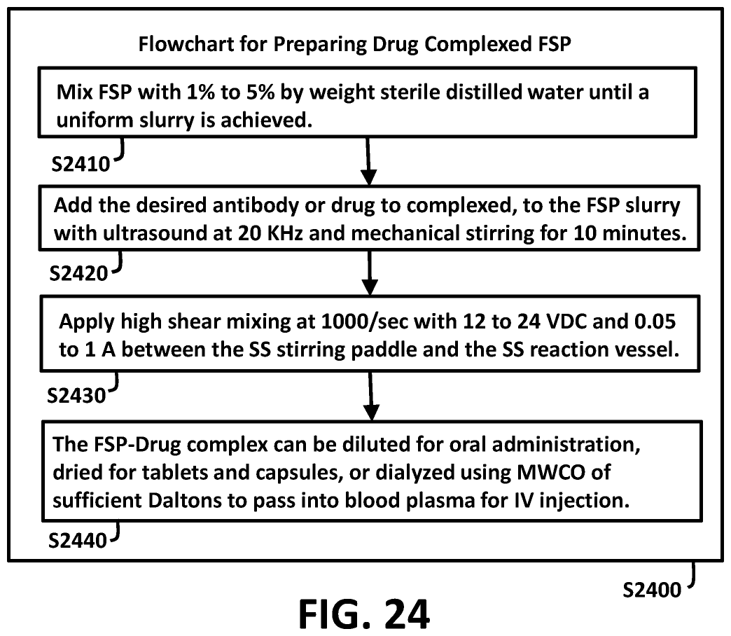

FIG. 24 is a flowchart showing exemplary steps to prepare drug complexed FSP. In step S2410, fullerene sodium phosphonate is mixed with 1% to 5% by weight of sterile distilled water until a uniform slurry is achieved. In step S2420, a desired antibody or drug to be complexed with FSP is added to the slurry mixture, and this slurry mixture is then ultrasonicated with the application of ultrasound at 20 KHz together with mild or slow mechanical stirring for 10 minutes to ensure an homogeneous dispersion. In step S2430, high shear mixing is then applied to the slurry mixture at 1000/sec with 12 to 24 VDC and 0.05 to 1 ampere between a pharmaceutical grade stainless steel stirring paddle and a stainless-steel reaction vessel. The high shearing action generates negative charges in solution which collect on the fullerene. The applied voltage and current can be adjusted based on the distance and geometry of the conductive paddle vanes to the conductive inside diameter of the reactive shear mixing chamber or vessel, and with respect to the dielectric properties of the antibody or drug to which the complex is to form with fullerene sodium phosphonate. The amount of time required for this process of reactive chemo-electric shear mixing, also known as chemo-electric reaction shear mixing, can be improved by reducing the amount of water added in step S2410 at the expense of requiring more torque on the shearing paddles. For most cases a process time of 20 to 30 minutes is sufficient at room temperature, however in some cases the process duration can be reduced by using elevated temperatures depending on the known thermal stability limit of the drug to be processed. In step S2440, the fullerene sodium phosphonate-drug complex can be diluted for oral administration, dried for tablets and capsules, or dialyzed using a MWCO of sufficient Daltons to selectively pass the resultant drug-FSP complex for the purpose of ensuring purity to meet a molecular weight in blood plasma for IV injection. The FSP-drug complex can require an expiration date or require refrigeration to enable the preservation of the synthesized drug-FSP complex to ensure the delivery efficacy on administration to a patient. FSP-drug stability will depend on such factors as the nature of the formed molecular complex, the geometry of the drug molecule or antibody, and the number of FSP molecules complexed with the drug molecule. For example, any number of faradic counter-ion charge complexes can be formed between the positive sodium cations of FSP and negatively charged (anionic) groups within the molecular structure of the therapeutic drug molecule.

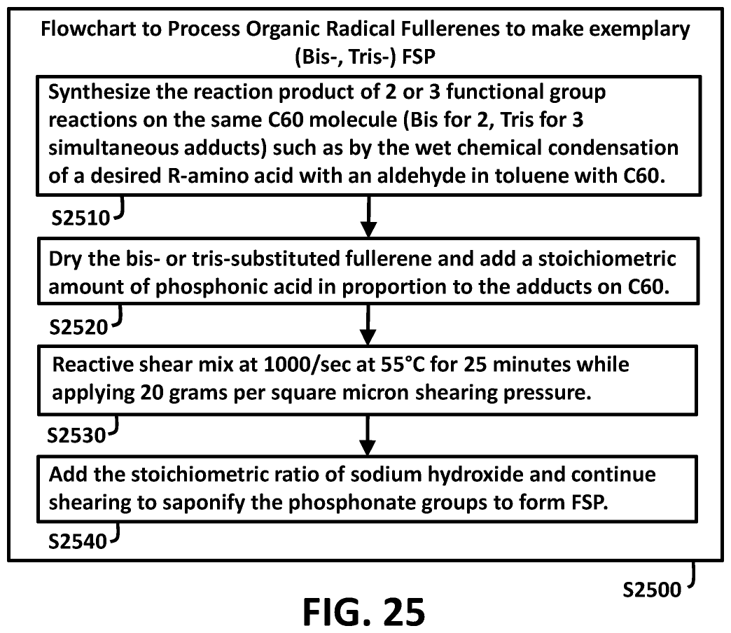

FIG. 25 is a flowchart showing exemplary steps to prepare fullerene sodium phosphonate having organic radical groups between the fullerene and the sodium phosphonate. In optional step S2510, an exemplary wet chemical condensation of a desired R-amino acid with an aldehyde and a C60 is performed in a solvent matrix such as toluene in accordance with the well-known Prado reaction. All traces of solvent must be removed after this reaction is completed. This step may also be accomplished using, for example, the well-known Bingel reaction for C60. Such organic radical substituted C60 products may not have pendant phosphonate groups. In the alternative to performing step S2510, the organic radical substituted C60 products may be purchased commercially.

In step S2520 the bis- or tris- or multiply substituted organic radical fullerene is dried and a stoichiometric amount of phosphonic acid is added in proportion to the organic radical adducts on C60 with which these are to react. In step S2530 reactive shear mixing is performed at 1000/sec at 55° C. for 25 minutes while applying about 20 grams per square micron shearing pressure to form the phosphonate groups at the organic radicals bonded to the C60. In step S2540, a stoichiometric ratio of sodium hydroxide is added to substantially neutralize the phosphonate groups with sodium cations and the reactive shear mixing process is continued to achieve saponification, thereby forming the type of fullerene sodium phosphonate that is provided with an organic radical (R) that is bonded between the fullerene and the sodium phosphonate.

As variations, combinations and modifications may be made in the construction and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments but defined in accordance with the foregoing claims appended hereto and their equivalents.

What is claimed is:

1. An antimicrobial nanoparticle composition comprising:
a buckminsterfullerene (C60) bonded to sodium phosphonate to form a fullerene sodium phosphonate, wherein
the Na+ ions of the sodium phosphonate are proximal to oxygen atoms bonded to phosphorus atoms and are reversibly pi-cation bonded with the C60 molecular structure, and
a hopping distance between the sodium phosphonate and the C60 is less than 5 nanometers.

2. The antimicrobial nanoparticle composition of claim 1 further comprising a solvent, wherein the C60 sodium phosphonate is disposed in the solvent.

3. The antimicrobial nanoparticle composition of claim 2 wherein the solvent comprises a mixture of 70% glycerol and 30% polypropylene glycol by volume.

4. The antimicrobial nanoparticle composition of claim 1 further comprising a food grade solid carrier, wherein the fullerene sodium phosphonate is mixed with the food grade solid carrier.

5. The antimicrobial nanoparticle composition of claim 4 wherein the food grade solid carrier includes baking powder, baking soda, or a powdered sweetener to make a food product.

6. The antimicrobial nanoparticle composition of claim 4 wherein the fullerene sodium phosphonate mixed with the food grade solid carrier is disposed in a gelatin capsule or formed into a tablet.

7. The antimicrobial nanoparticle composition of claim 1 further comprising a blood plasma with the fullerene sodium phosphonate dissolved therein.

8. The antimicrobial nanoparticle composition of claim 1 further comprising a first organic functional group including a carbon atom that is bonded to at least one carbon atom of the C60, wherein the sodium phosphonate is bonded to the first organic functional group that is also bonded to the C60.

9. The antimicrobial nanoparticle composition of claim 8 further comprising a second organic functional group bonded to the C60, wherein the second organic functional group includes a second sodium phosphonate.

10. A method of curing, treating, or prophylactically avoiding cancer, valley fever, COPD, pneumonia, antibody-resistant bacterial infections in a subject, and to treat an absence of a missing sodium control protein in a muscular myopathy, comprising the step of:
administering to the subject a pharmaceutically effective amount of a composition including a buckminsterfullerene (C60) bonded to sodium phosphonate to form a fullerene sodium phosphonate, wherein the Na+ ions of the sodium phosphonate are proximal to oxygen atoms bonded to phosphorus atoms and are reversibly pi-cation bonded with the C60 molecular structure and a hopping distance between the sodium phosphonate and the C60 is less than 5 nanometers.

11. The method of claim 10 wherein the composition includes a food grade solid carrier including baking powder or baking soda, and the fullerene sodium phosphonate is disposed in the food grade solid carrier.

12. The method of claim 11 wherein the composition comprises a tablet, capsule, pill, powder, granule, or a liquid containing a dosage of 50 mg to 1000 mg of fullerene sodium phosphonate fullerene.

13. The method of claim 10 wherein administering the composition comprises administration by an intravenous, intramuscular, subcutaneous, intrathecal, intraperitoneal, topical, nasal, or oral route.

14. The method of claim 10 wherein administering the composition comprises administering an oral dosage including up to about 500 mg of the fullerene sodium phosphonate.

15. The method of claim 10 wherein administering the composition comprises administering an intramuscular, intravenous, or a subcutaneous dose of fullerene sodium phosphonate in an amount of from about 0.1 mg/Kg to about 5 mg/Kg.

16. The method of claim 10 wherein administering the composition comprises administering a volume of an inhalant in the form of an air dispersed nano aerosol, a water mist dispersed vapor, an air dispersed powder, an air dispersed dust, or an air dispersed aerosol.

17. A method of making an antimicrobial fullerene sodium phosphonate, the method comprising:
reacting phosphorous acid with C60 or polyhydroxylated C60 to produce a fullerene phosphonate; and
reacting the fullerene phosphonate with sodium hydroxide to form a fullerene sodium phosphonate.

18. The method of claim 17 wherein reacting the phosphorous acid with the C60 or the polyhydroxylated C60 is performed by reaction shear mixing.

19. The method of claim 17 wherein reacting the sodium hydroxide with the fullerene phosphonate is performed by reaction shear mixing.

20. The method of claim 17 wherein reacting the phosphorous acid with the C60 or the polyhydroxylated C60 is performed in the presence of sodium hydroxide such that reacting the fullerene phosphonate with sodium hydroxide to produce the fullerene sodium phosphonate occurs in a same processing step as reacting the phosphorous acid with the C60 or the polyhydroxylated C60.

21. The method of claim 17 wherein the C60 is a bis-C60 or a tris-C60.
